(19) United States
(12) Patent Application Publication
(10) Pub. No.: US 2006/0135925 A1
(43) Pub. Date: Jun. 22, 2006

(54) Diaper system that includes an applique

(75) Inventors: Steven M. Hurley, Neenah, WI (US); Heidi Hopkins, Neenah, WI (US); Kellie Marie Goodrich, Appleton, WI (US)

Correspondence Address: SCHWEGMAN, LUNDBERG, WOESSNER & KLUTH, P.A., P.O. BOX 2938, MINNEAPOLIS, MN, 55402, US

(73) Assignee: Kimberly-Clark Worldwide, Inc.

(21) Appl. No.: 11/016,463

(22) Filed: Dec. 17, 2004

(51) Int. Cl. A61F 13/15

(52) U.S. Cl. 604/385.01

(57) ABSTRACT

The present invention relates to a system that includes a package and a plurality of diapers that are within the package. The system further includes an appliqué that is within the package. The appliqué is adapted to be detachably adhered to each of the plurality of diapers in the system. The appliqué may initially be placed on one of the diapers and when the original diaper is discarded, the appliqué may then be transferred to another newly worn diaper in order to promote interaction between a child and the diapers that are used during multiple diaper changes. In some embodiments, the appliqué includes a first graphic and at least one of the diapers in the system includes a second graphic. The first graphic and the second graphic may form a third graphic when the appliqué is detachably adhered to an appropriate location on the diapers that include the second graphic.

### FIELD

Some embodiments of the invention relate to a system that includes diapers, and in particular to a system that includes an appliqué which may be detachably adhered to diapers in the system.

### BACKGROUND

A typical caregiver performs several diaper changes per day. It is usually easier to change diapers when a child is able to interact with the diapers as part of the diaper changing process.

One known method of facilitating interaction between a child and diapers is to place graphics on the diapers that serve to stimulate the child. The graphics often enhance dialog between the caregiver and the child during diaper changes.

One drawback with placing graphics on diapers is that the graphics are typically of low quality due to the constraints which are associated with economically manufacturing diapers. As an example, it is often difficult to place high quality graphics and/or certain types of graphics on diapers because diapers are typically manufactured at extremely high speeds using materials that readily absorb the inks, dyes and other materials which form the graphics.

Another drawback with placing graphics on diapers is that a child is typically unable to translate any interaction from one diaper to another during diaper changes. There is little or no interaction between diapers because the used diapers get discarded as part of the diaper changing process.

### SUMMARY OF THE INVENTION

The present invention relates to a system that aids care providers when they need to perform multiple diaper changes per day. The system makes the diaper changing process more interactive between children and diapers, especially from one diaper change to another.

In one aspect, the system includes a package and a plurality of diapers that are within the package. The system further includes an appliqué that is within the package and is adapted to be detachably adhered to each of the plurality of diapers in the system. The appliqué may be placed on one of the diapers in the system which is initially worn by a child. When the original diaper is changed and then discarded, the appliqué may then be transferred to another newly worn diaper thereby facilitating interaction between the child and the diapers in the system from one diaper change to another.

In some embodiments, the appliqué may be formed of several decorative colors and/or may include a textured surface to provide aesthetic and/or tactile stimulation. The appliqué may also have a geometric shape (e.g., circle, square, triangle, and hexagon) or an animal shape (e.g., lion, elephant and human).

In another aspect, the appliqué may include a first graphic and at least one of the diapers in the system may include a second graphic. The first graphic and the second graphic may form a third graphic when the appliqué is detachably adhered to an appropriate location on the diapers that include the second graphic. In some embodiments, each of the diapers in the system may include a unique second graphic such that the first graphic and each unique second graphic form a distinctive third graphic when the appliqué is detachably adhered to an appropriate location on each of the diapers.

In another aspect, the system may include a plurality of appliqués that are adapted to be detachably adhered to each of the plurality of diapers. In some embodiments, each appliqué is different from at least one of the other appliqués in the system in at least one feature (i.e., color, shape, texture). In addition, the first and second graphics may be designed such that the first graphic covers a portion of the second graphic to form the third graphic.

The purposes and features of the present invention will be set forth in the description that follows. Additional features of the invention may be realized and attained by the product and processes particularly pointed out in the written description and claims hereof, as well as from the appended drawings.

It is to be understood that both the foregoing general description and the following detailed description are exemplary and are intended to provide further explanation of the invention claimed. The accompanying drawings, which are incorporated in and constitute part of this specification, are included to illustrate and provide a further understanding of the invention.

### BRIEF DESCRIPTION OF THE DRAWINGS

The present invention will be more fully understood, and further features will become apparent, when reference is made to the following detailed description and the accompanying drawings. The drawings are merely representative and are not intended to limit the scope of the claims. Like parts depicted in the drawings are referred to by the same reference numerals.

FIG. 1 is a schematic perspective view illustrating an example system of the present invention.

FIG. 2 illustrates part of the system shown in FIG. 1 where the appliqué in the system is transferred between diapers in the system.

FIGS. 3-10 are schematic perspective views illustrating example appliqués that may be used in any of the example systems described herein.

FIG. 11 is a schematic perspective view illustrating another example system of the present invention.

FIG. 12 illustrates part of the system shown in FIG. 11 where the appliqué in the system is transferred between diapers in the system.

FIG. 13 is a schematic perspective view illustrating still another example system of the present invention.

FIG. 14 illustrates part of the system shown in FIG. 13 where the appliqué in the system is transferred between diapers in the system.

FIG. 15 is a schematic perspective view illustrating yet another example system of the present invention.

FIG. 16 illustrates part of the system shown in FIG. 15 where the appliqués in the system are transferred between diapers in the system.

FIG. 17 illustrates some example appliqués that may be used in any of the example systems described herein where the appliqués are mounted onto a diaper.

### DETAILED DESCRIPTION OF THE INVENTION

In the following detailed description, reference is made to the accompanying drawings, which show specific embodiments in which the invention may be practiced. These embodiments are described in sufficient detail to enable those skilled in the art to practice the invention. It is to be understood that other embodiments may be utilized and structural changes made, such that the following detailed description is not to be taken in a limiting sense.

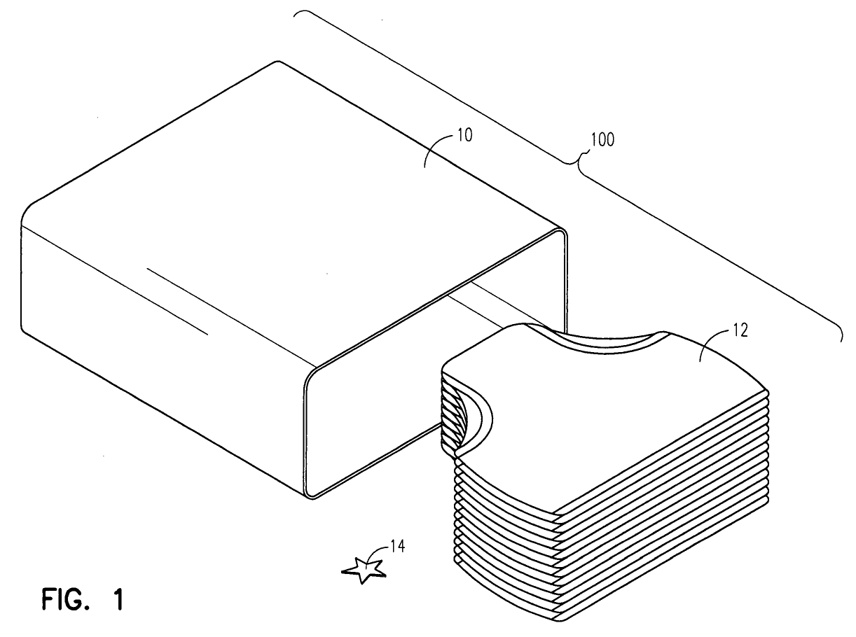

FIG. 1 illustrates a system 100 that includes a package 10 and a plurality of diapers 12 that are within the package 10. The system 100 further includes an appliqué 14 that is within the package 10. The appliqué 14 is adapted to be detachably adhered to each of the plurality of diapers 12. It should be noted that the system 100 may be provided to a care giver such that the appliqué 14 is detachably adhered to one of the diapers 12 within the package 10 or loosely held within the package 10.

The system 100 may be used by a care giver to perform multiple diaper changes. Since the appliqué 14 can be detachably adhered to each of the diapers 12 in the system 100, the appliqué may be placed onto one of the diapers 12 that is initially being worn a child in order to provide interaction between the child and the diaper 12.

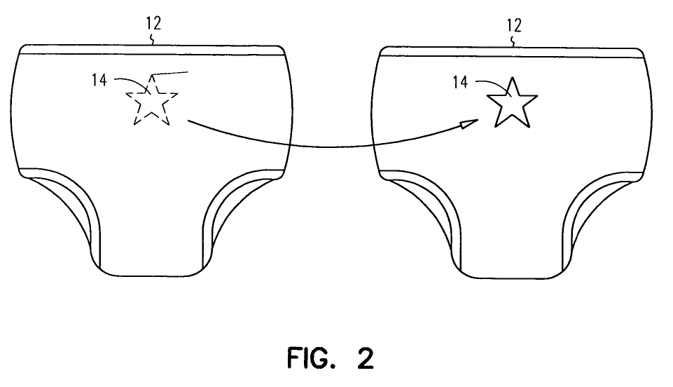

As shown in FIG. 2, when the original diaper 12 is changed and then discarded, the appliqué 14 may then be transferred by the care giver and/or the child to another newly worn diaper 12. The interaction between the child and the appliqué 14 that results from transferring the appliqué 14 may facilitate bonding between the child and the diapers 12. If a child feels a bond with the diapers 12, it may become easier for a care giver to gain the child's cooperation during diaper changes.

Any type of package 10 may be used in system 100. Some example packages 10 include containers, holders, cans, jars, racks, bags, pouches, cubbies, dispensers, spindles, cores, cupboards, closets, clips, cabinets, crates, trunks, totes and/or tubs (among others).

The package 10 may be disposable or reusable and may be plain or decorated in some manner. In addition, the package 10 may be used as a shipping container or to perform some other function.

Any type of diaper 12 may be used in system 100 or any of the systems described herein. Some example diapers include newborn diapers, premature baby diapers, overnight diapers, diapers for adults and diapers of various other sizes. Other example diapers include specialized diapers, such as diapers for urine absorption, feces absorption, blood absorption, sweat absorption, menses absorption and diapers that include a lotion or medication. The diapers may also be swim pants, training pants, diaper pants and absorbent underpants (among others).

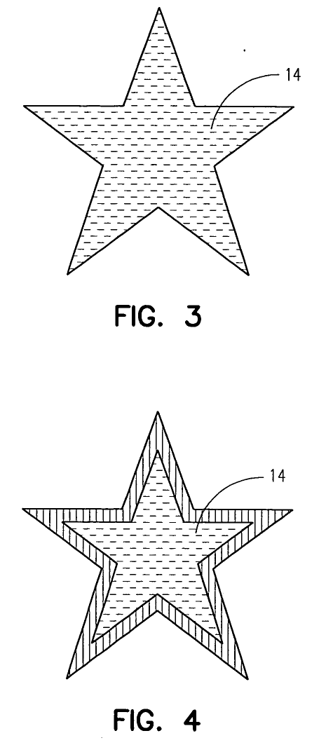

As used herein, an appliqué 14 is something that may be detachably adhered to each of the diapers 12. The appliqué 14 may be any type of appliqué 14 that is known now or discovered in the future. FIG. 3 shows that the appliqué 14 may be one color and FIG. 4 shows that the appliqué 14 may be formed of two or more decorative colors.

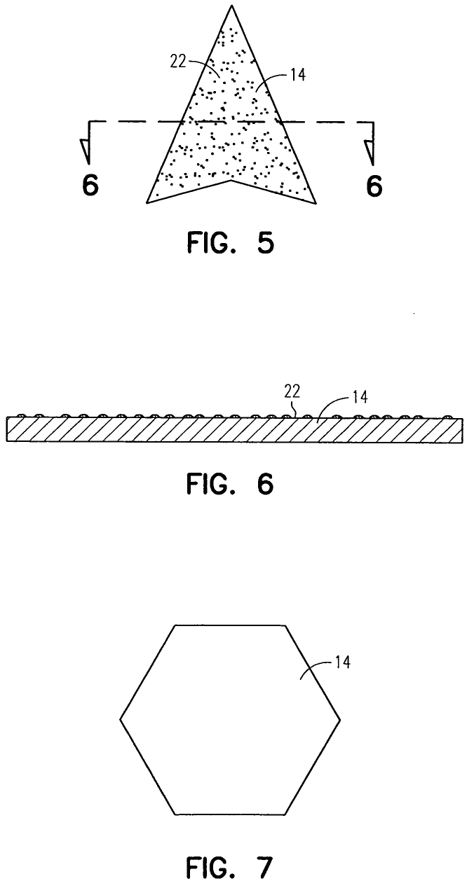

FIG. 6 is a section view of FIG. 5 taken along line 6-6 in FIG. 5. As shown most clearly in FIG. 6, the appliqué 14 may include a textured surface 22 that provides aesthetic and/or tactile stimulation between the child and the appliqué 14. In some embodiments, the appliqué 14 may include simulated stones, jewels or gems to provide additional aesthetic and/or tactile stimulation. In addition, the appliqué 14 may emit a fragrance or aroma (e.g., lilac, strawberry, chocolate).

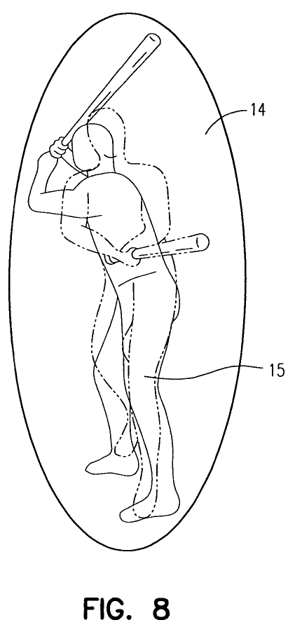

In some embodiments, the appliqué 14 may be a geometric shape such as a circle, square and triangle. FIG. 7 shows an example hexagon shape for appliqué 14.

FIG. 8 shows an example appliqué 14 that includes a hologram 15 which appears to change image depending on the orientation of how the hologram 15 is viewed by someone examining the appliqué 14. The hologram 15 is one example of a graphic that is difficult to place directly on a diaper because of the constraints that are associated with manufacturing diapers. The hologram 15 is more readily formed on the appliqué 14.

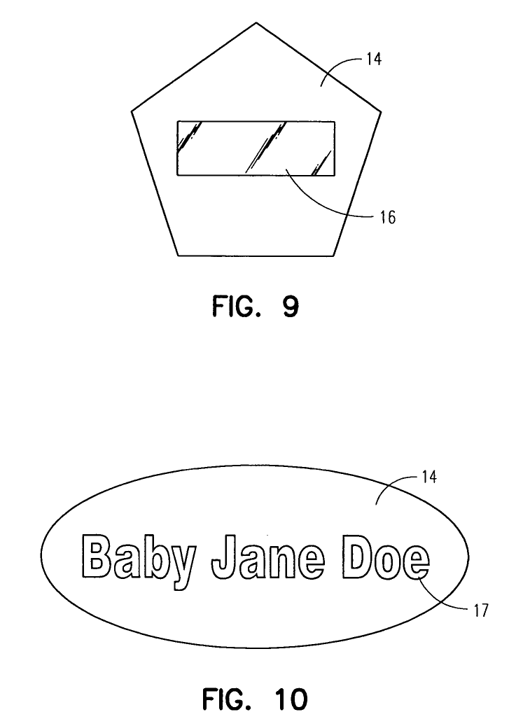

As shown in FIG. 9, the appliqué 14 may include a reflective section 16. It should be noted that the reflective section 16 may be any shape and size. In some embodiments, the entire appliqué 14 may be formed of a reflective material.

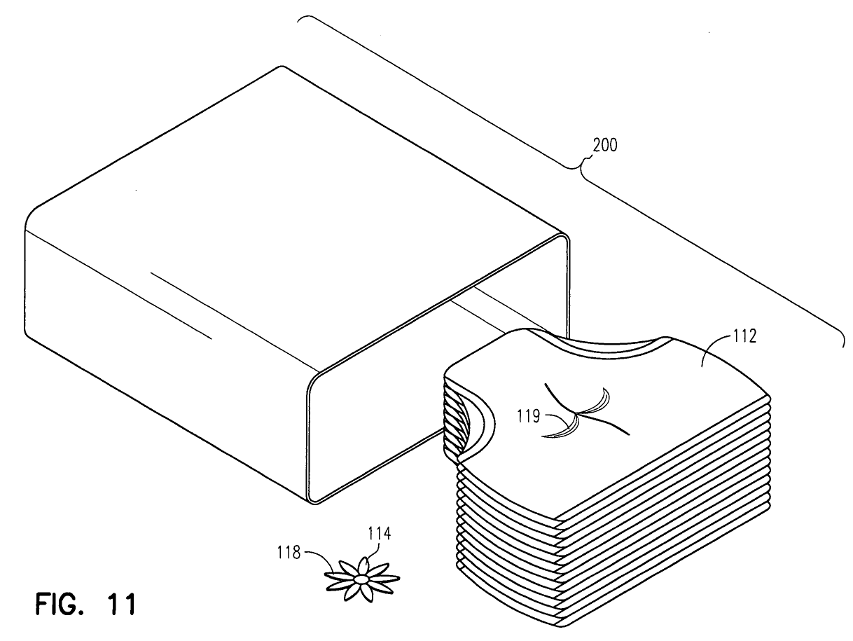

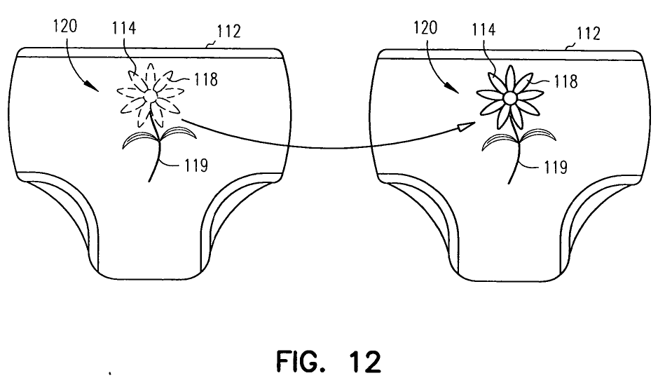

FIG. 10 illustrates that the appliqué 14 may include one or more graphics that contain alphanumeric information 17. In some embodiments, the alphanumeric information 17 may include identification information related to a child that is to wear the diapers 12 and/or the parents of the child that is to wear the diapers 12 (e.g., parent's and/or child's names). In addition, the alphanumeric information 17 may include instructions to a care giver as to how to change a diaper.

It should be noted that any type of alphanumeric information 17 may be included on appliqué 14. As an example, the alphanumeric information 117 may identify the day of the week (e.g., Monday, Tuesday, etc.) when a particular diaper is to be used.

The appliqué 14 may be detachably adhered to the diapers 12 through any method that is known now or discovered in future as long as the appliqué 14 may be transferred from one diaper 12 to another. As an example, the appliqué 14 and the plurality of diapers 12 may form a hook-and-loop type fastener when the appliqué 14 is detachably adhered to one of the plurality of diapers 12. Some of the other example methods that may be used to detachably adhere the appliqué 14 to the diapers 12 include using magnets, snaps and adhesives.

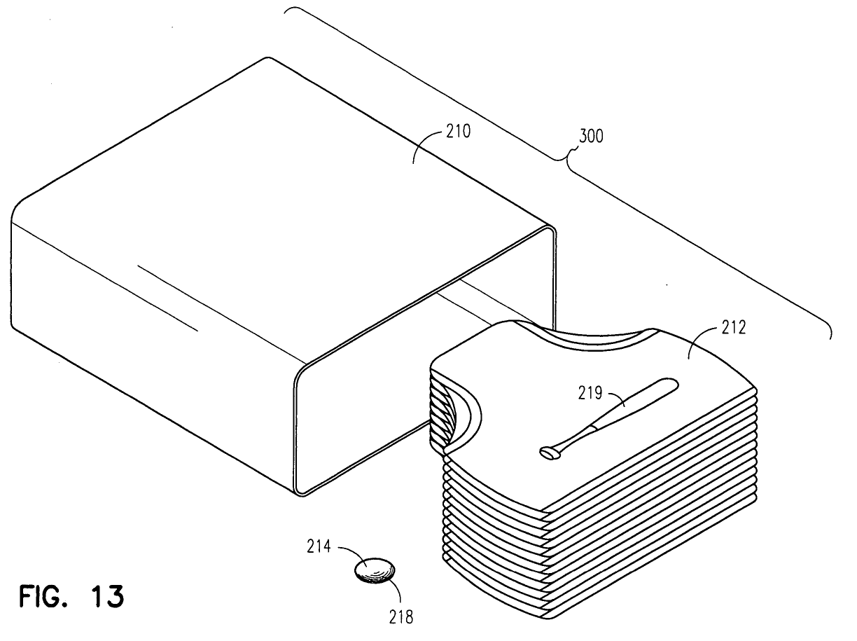

FIG. 11 illustrates another example system 200 of the present invention. The system 200 includes a package 110 and a plurality of diapers 112 that are within the package 110. The system 200 further includes an appliqué 114 that is within the package 110 and is adapted to be detachably adhered to each of the plurality of diapers 112 within the system 200.

In the example embodiment illustrated in FIG. 11, the appliqué 114 includes a first graphic 118 and at least one of the diapers 112 in the system 200 includes a second graphic 119. It should be noted that one, some or all of the diapers 112 in the system 200 may include the second graphic 119.

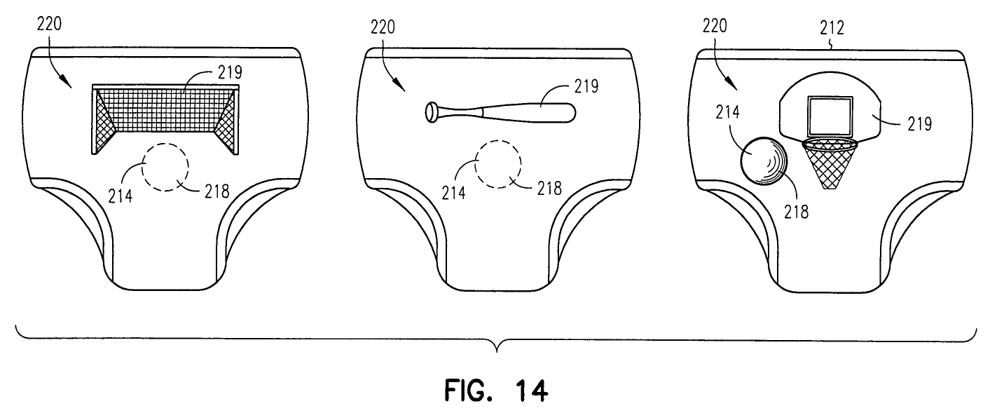

As shown in FIG. 12, the first graphic 118 and the second graphic 119 may form a third graphic 120 when the appliqué 114 is detachably adhered to an appropriate location on the diapers 112 that include the second graphic 119. FIG. 12 also shows the appliqué 114 as the appliqué 114 is transferred from one diaper 112 in the system 200 to another diaper 112 in the system 200.

The first graphic 118 and the second graphic 119 may be any type of graphic that is known now or discovered in the future. It should be noted that the first graphic 118 may be the same as the second graphic 119 or different than the second graphic 119. In addition, the first graphic 118 may form part of, or all, of the appliqué 114 depending on the design considerations that are associated with the system 200.

FIG. 13 illustrates another example system 300 that includes a package 210 and a plurality of diapers 212 that are within the package 210. The system 300 further includes an appliqué 214 that is within the package 210. The appliqué 214 is adapted to be detachably adhered to each of the plurality of diapers 212 in the system 300.

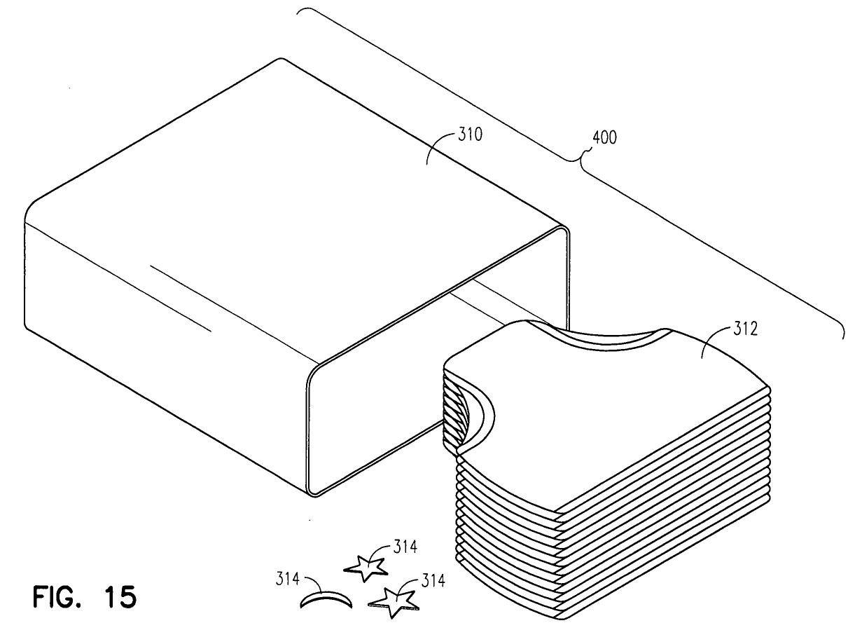

In the example embodiment illustrated in FIG. 13, the appliqué 214 includes a first graphic 218 and each of the diapers 212 includes a second graphic 219. As shown in FIG. 14, the second graphic 219 on each diaper 212 is different from the second graphic 219 on each of the other diapers 212 in the system 300. Therefore, the first graphic 218 and each unique second graphic 219 form a distinctive third graphic 220 when the appliqué 214 is detachably adhered to an appropriate location on each of the diapers 212. FIG. 14 also shows the appliqué 214 as the appliqué 214 is transferred from one diaper 212 in the system 300 to another diaper 212 in the system 300.

FIG. 15 illustrates another example system 400 that includes a package 310 and a plurality of diapers 312 that are within the package 310. The system 400 further includes a plurality of appliqués 314 that are within the package 310 (three appliqués 314 are shown in FIG. 15). Each of the appliqués 314 is adapted to be detachably adhered to each of the plurality of diapers 312 in the system 400.

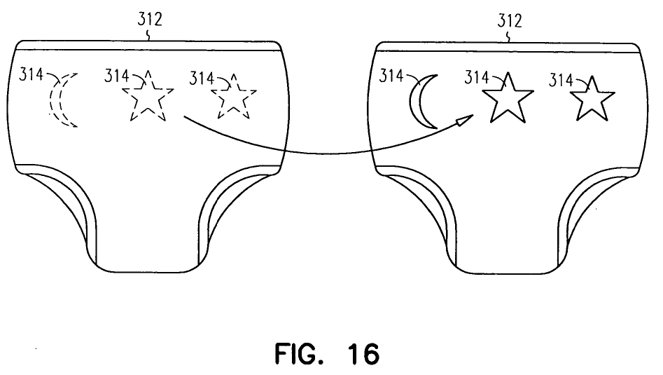

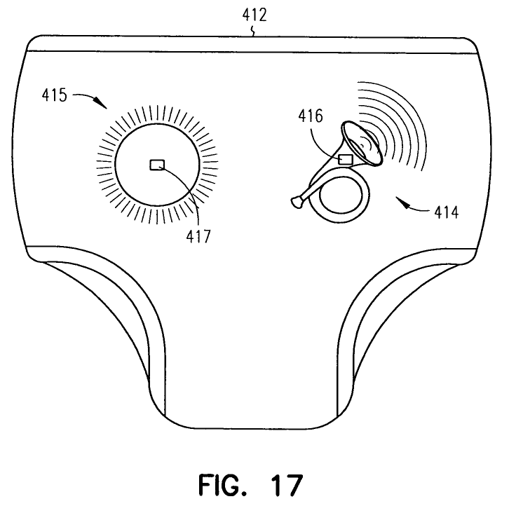

In the example embodiment illustrated in FIG. 15, each of the appliqués 314 is different from at least one of the other appliqués 314 in the system 400. FIG. 16 shows the appliqués 314 as the appliqués 314 are transferred from one diaper 312 to another diaper 312 in order to facilitate interaction between the child and each diaper 312 in the system 400.

FIG. 17 illustrates some example additional appliqués 414, 415 that may be used in any of the example systems described herein. The appliqués 414, 415 are mounted onto a diaper 412. One the appliqués 414 includes a mechanism 416 that produces sound and the other appliqué 415 includes a mechanism 417 that produces light. It should be noted that appliqués are contemplated which include one or more mechanisms that produce light and/or sound. The ability of an appliqué to produce light and/or sound may further facilitate interaction between a child, the appliqué and each diaper in the system.

The system described herein may facilitate multiple diaper changes per day by promoting interaction between a child and the diapers that are used during multiple diaper changes. The system also allows a child to translate interaction from one diaper to another during diaper changes.

FIGS. 1-17 are representational and are not necessarily drawn to scale. Certain proportions thereof may be exaggerated, while others may be minimized.

While the invention has been described in detail with respect to the specific aspects thereof, it will be appreciated that those skilled in the art, upon attaining an understanding of the foregoing, may readily conceive of alterations to, variations of, and equivalents to these aspects which fall within the spirit and scope of the present invention, which should be assessed accordingly to that of the appended claims.

We claim:

1. A system comprising:
a package;
a plurality of diapers within the package; and
an appliqué within the package, the appliqué being adapted to be detachably adhered to each of the plurality of diapers.

2. The system of claim 1 wherein the appliqué is one color.

3. The system of claim 1 wherein the appliqué is multiple colors.

4. The system of claim 1 wherein the appliqué is a geometric shape.

5. The system of claim 1 wherein the appliqué includes a hologram.

6. The system of claim 1 wherein the appliqué includes a reflective section.

7. The system of claim 1 wherein the appliqué includes a textured surface.

8. The system of claim 1 wherein the appliqué includes alphanumeric information.

9. The system of claim 1 wherein the appliqué and the plurality of diapers form a hook and loop type fastener when the appliqué is detachably adhered to one of the plurality of diapers.

10. The system of claim 1 wherein the appliqué includes a first graphic.

11. The system of claim 10 wherein at least one the diapers includes a second graphic.

12. The system of claim 111 wherein the first graphic and the second graphic form a third graphic when the appliqué is adhered to an appropriate location on the diapers that include the second graphic.

13. The system of claim 12 wherein the first graphic and the second graphic form a third graphic when the appliqué is adhered to the diapers over at least a portion of the second graphic on the diapers.

14. The system of claim 10 wherein each of the diapers includes a second graphic.

15. The system of claim 14 wherein the first graphic and the second graphic form a third graphic when the appliqué is detachably adhered to an appropriate location on each of the diapers.

16. The system of claim 15 wherein the first graphic and the second graphic form a third graphic when the appliqué is adhered to the diapers over at least a portion of the second graphic on the diapers.

17. The system of claim 14 wherein each of the second graphics on the diapers is different from each of the other second graphics on the diapers such that the first graphic and each second graphic form a distinctive third graphic when the appliqué is detachably adhered to an appropriate location on the diapers.

18. The system of claim 1 further comprising at least one additional appliqué within the package, wherein each of the additional appliqués is adapted to be detachably adhered to each of the plurality of diapers.

19. The system of claim 18 wherein each appliqué within the package is distinctive from at least one of the other appliqués within the package.

20. The system of claim 1 wherein the appliqué includes a mechanism that produces sound.

21. The system of claim 1 wherein the appliqué includes a mechanism that produces light.

22. The system of claim 1 wherein the appliqué is detachably adhered to one of the diapers within the package.

23. A system comprising:
a package;
an appliqué within the package, the appliqué including a first graphic;
a plurality of diapers within the package, each of the diapers including a second graphic such that the first graphic and the second graphic form a third graphic when the appliqué is detachably adhered to an appropriate location on each of the diapers.

24. The system of claim 23 further comprising at least one additional appliqué within the package, wherein each of the additional appliqués is adapted to be detachably adhered to each of the plurality of diapers.

25. The system of claim 23 wherein the appliqué includes alphanumeric information.
